(19) United States
(12) Patent Application Publication
(10) Pub. No.: US 2010/0099942 A1
(43) Pub. Date: Apr. 22, 2010

(54) METHOD AND APPARATUS FOR ELECTROMAGNETIC HUMAN AND ANIMAL IMMUNE STIMULATION AND/OR REPAIR SYSTEMS ACTIVATION

(76) Inventor: Lucas Portelli, Boulder, CO (US)

Correspondence Address: SHERIDAN ROSS PC, 1560 BROADWAY, SUITE 1200, DENVER, CO, 80202, US

(21) Appl. No.: 12/254,273

(22) Filed: Oct. 20, 2008

(51) Int. Cl. A61N 2/02; A61N 1/00

(52) U.S. Cl. 600/13; 607/2

(57) ABSTRACT

A method and device that can deliver a time-varying electric field non-invasively inside of biological tissue and/or biological fluid such as blood and/or lymph and/or synovial fluid and/or interstitial fluid and/or other fluids in-vivo and with magnitudes that do not cause a tolerable amount of neural motor and sensory action potential generation and frequency components below the megahertz range by means of the placement of more than one electrode on the skin with or without some intermediate biological, synthetic, or natural agent or substance between the electrode and the skin and/or by induction by means of a time-varying magnetic field or both with the novel purpose of activation and/or potentiation and/or normalization and/or regulation and/or stimulation and/or signaling and/or increase and/or regulation of the alertness and/or self tuning of the immune system or any of its parts or components directly or indirectly, locally and/or globally with the purpose of pathogen load reduction and/or reduction of the disease symptoms and/or clinical improvement and/or tumor size reduction and/or malignancy reduction or amelioration and/or improvement of the effects of an autoimmune disorder or any other related condition that could be treated or remised by its effects.

### FIELD OF THE INVENTION

The present invention is generally directed towards methods for stimulating and/or activating and/or potentiating and/or normalizing and/or regulating and/or signaling and/or the immune system and/or repair systems or any of its parts or components directly and/or indirectly, and more particularly to stimulating and/or activating and/or potentiating and/or normalizing and/or regulating and/or signaling and/or the immune system and/or repair systems or any of its parts or components directly and/or indirectly, trough the use of electromagnetic stimuli

### BACKGROUND OF THE INVENTION

It is known that electromagnetic signals may have a myriad of difficultly predictable or measurable effects when applied to biological systems. This is due mainly because of the biological systems' innate complicated nature and because of the broad spectrum of characteristics that an electromagnetic signal can have. In the case of biological systems, they can basically be represented by a convoluted system of feedback loops (some of them that might not even been identified yet) that present non-linearity, time dependency as some of their properties. In the case of electromagnetic signals they present characteristics such as amplitude, frequency components, exposure and non-exposure (rest) time, sequence or time dependency, as well as the exposure system geometry and relative position, etc. all of which, as well as the biological systems' characteristics, can vary by several orders of magnitude. All this makes the study and understanding of their interaction not an easy task. Nevertheless, in the last 30 years, extensive empirical and theoretical work has been done regarding the healing effects of electromagnetic fields, from where some methods and apparatuses have derived. Some of these are of some interest to the scope of this invention and are here presented.

One of the approaches proposed for effecting biological systems with electromagnetic signals non-invasively has been the Ion Cyclotron Resonant Frequency (ICRF) method, where an electromagnetic field is applied in conjunction with a static magnetic field in order to generate changes in ionic transport in cells part of a living organism. Liboff et al. (U.S. Pat. No. 4,818,697) Techniques for enhancing the permeability of ions trough membranes—are some of the first describing the use of this method. Departing from this same principle, Baylink (U.S. Pat. No. 5,195,940) Method for increased production of growth factor in living tissue using an applied fluctuating magnetic field—Describes the increase in production of growth factor by the application of a static and a dynamic magnetic field related to the ICRF. Also, Liboff et al. (U.S. Pat. No. 5,290,409) Methods and apparatus for regulating transmembrane ion movement utilizing selective harmonic frequencies and simultaneous multiple regulation—describe the same principle but approached in a way that harmonics of the ICRF are used for the same purpose. Other previous art related to ICRF is done by Jacobson (U.S. Pat. No. 6,004,257) Method for ameliorating the aging process and the effects thereof utilizing electromagnetic energy—Describing another application related to the ICRF with magnitudes of the AC field between 10⁻²⁰ and 10⁻⁶ gauss and 0-10¹⁴ Hz (preferably below 1000 Hz) utilizing a time dependant signal. The same is done by Jacobson (U.S. Pat. No. 6,099,459) Magnetic Field generating device and method of generating and applying a magnetic field for treatment of specified conditions—where an apparatus for the application of an electromagnetic signal ranging from DC to 20 Mhz and 10 m to 10 atto gauss with a Helmholtz coil is described, and by Cuppen (U.S. Pat. No. 7,008,369) Apparatus and method for electromagnetic therapy—Where the use of two different and approximately perpendicular fields: a static magnetic field and a time varying electromagnetic field being the static the earth's magnetic field or other artificially created field (ICRF Approach) is described to be used with the purpose of influencing several types of ions directly in order to stimulate physiological processes among them the stimulation of the immune system. The AC signal in this case is composed of a combination of several frequencies that are not necessarily multiples and not interrelated in amplitude (in other words not necessarily pulsed in the time domain) in the range of 0.1 to 40 kHz according to the ICRF.

An alternative to the ICRF approach with similar ends is presented by Muntermann (U.S. Pat. No. 6,558,311) Device for treatment with magnetic fields and similarly in Muntermann (U.S. Pat. No. 6,461,289) Device for magnetic field therapy—where an apparatus for exposing a portion of a biological body to both a static and a changing magnetic field in order to produce nuclear spin resonances (NSR) is described.

Notice that the previous art presented claim or imply the non-invasive activation and/or potentiation and/or normalization and/or regulation and/or stimulation, etc. of the immune system or any of its parts or components directly or indirectly, locally and/or globally, need of the application or presence of two signals in the same area (being one of them static and if it is the earth's magnetic field then the azimuth orientation of the device plays an important role) and the other dynamic in order to take advantage of the claimed effects of the ICRF or NSR.

Another approach that does not imply the utilization of simultaneous perpendicular fields being one of them static and both applied to the same area (ICRF or NSR) for effecting biological systems has been presented by Ryaby et al. (U.S. Pat. No. 4,315,503) Modification of the growth, repair and maintenance behavior of living tissues and cells by a specific selective change in electrical environment—Where a method for regenerating tissue by using electromagnetic fields by inducing currents in the order of 0.1-10 uA/cm2 and by using combinations of Low frequencies is described. Markoll (U.S. Pat. No. 5,453,073) Apparatus for treating of diseased body organs with magnetic field therapy And Markoll (U.S. Pat. No. 6,119,631) Coil cabinet for treating animals with magnetic field therapy—Describes an apparatus for the application of a pulsed magnetic field on a human body with therapeutic intentions being its signal characteristics 20 gauss and 1 to 30 cycles per second and describing a cabinet for animal treatment as well. Also Rey (U.S. Pat. No. 6,497,648) Device for applying electromagnetic therapy—Describes a device that generates a pulsing field for treating bone and tissue diseases. Also Dissing et al. (U.S. Pat. No. 6,561,968) Method and apparatus for stimulating/modulating biochemical processes using pulsed electromagnetic fields—Describe an apparatus comprised of magnetic coils arranged in a honeycomb configuration for the delivery of a pulsed electromagnetic field inside of a living organism in order to stimulate growth while avoiding the generation of action potentials. The signal properties are 0.05-0.1 T and 1-300 Hz for a period of 15 minutes to more than 2 days of continuous application.

It is also known that Electromagnetic fields have the ability of creating a stress to a living organism due to its effect on charged particles and that stress invites the immune and repair systems of an organism to actuate in order to contain what primarily caused this stress which can also be potentially used as a healing aid. Litovitz (U.S. Pat. No. 6,856,839) Use of electromagnetic fields in cancer and other therapies—Describes an apparatus for delivery of electromagnetic fields in order to create enough stress to treat diseases and conditions by regulating the appearance or stress proteins. The signal magnitude is between 2 and 2000 uT and 10 to 15 GHz where the signal is applied to the whole body or to the diseased part. A similar approach has been described by Irion et al. (U.S. Pat. Application No. 20020072646) Use of magnetic fields to enhance immune system performance—Where the use of Low energy alternating current magnetic fields to induce genes that regulate cellular stress response and increase the production of beneficial gene products causing little or no damage to the host organism for enhancing the immune system, maintaining/improving growth hormone production, immune system function, cellular insulin sensitivity, slowing or reversing the effects of aging in the circulatory system and preventing the on-set or progression of Alzheimer and Parkinson diseases is described. The signal is applied for 1 minute to 24 hours resting for 1 minute to 1 week at 0.001 to 2 Gauss. Notice that the electric field induced within the tissue will be variable and depend upon the frequency applied in the AC signal. Direct activation of the immune system related art has been proposed by Baugh (U.S. Pat. No. 7,338,431) Method and apparatus to stimulate the immune system of a biological entity—Describes the application of pulsed magnetic energy into the biological entity environment in order to activate the immune system.

Notice that some of the previous art presented claim or imply the non-invasive activation and/or potentiation and/or normalization and/or regulation and/or stimulation, etc. of the immune system or any of its parts or components directly or indirectly, locally and/or globally, rely on the application of magnetic field intensity not grater than a few mili-Teslas.

Blood borne viral infections are difficult to treat and difficultly curable after a living organism becomes infected. The same happens with fungal or bacterial infections since all of these might remain dormant within the organism and/or mutate with ease. Infections might also be sustained and enhanced by the presence of parasites since usually these need of a microbial infection in its life cycle. This, in combination with the fact that re-infection among individuals of a population is also present, leads to a inescapable need for constant reinvention and rediscovery of chemical substances capable of treat or ameliorate the pathogen proliferation and symptoms of its presence. Therefore, the existence of alternative therapeutics against pathogen related ailments are highly desirable. In the case of cancers & other related ailments this is also true since these cells seem to be able to reprogram or adapt to new environmental conditions with ease.

Pathogen direct inactivation by an electromagnetic field that creates biocompatible currents related art has been presented by Kaali et al. (U.S. Pat. No. 5,139,684) Electrically conductive methods and systems for treatment of blood and other body fluids and/or synthetic fluids with electric forces—Where an invasive dialysis-like approach to inactivate the virus, bacteria, parasites or fungus contained in the blood or other fluids by the action of an electric current with magnitudes in the order of 1 uA/cm2 to 1 mA/cm2 created by an electric potential of 0.2-12V within a chamber is described. Also, Kaali et al. (U.S. Pat. No. 5,185,086) Method and system for treatment of blood and/or other body fluids and/or synthetic fluids using combined filter elements and electric field forces, Kaali et al. (U.S. Pat. No. 5,188,738) Alternating current supplied electrically conductive method and system for treatment of blood and/or other body fluids and/or synthetic fluids with electric forces and Byrne et al.(U.S. Pat. No. 5,352,192) Medical Device—Describe similar invasive approaches.

Invasive approaches are not very desirable for several reasons. The invasive procedure creates inevitable damage when it is installed which increases the possibility of triggering more local infections. Also, the invasive components must have a regular maintenance protocol in order to assure its good functioning which usually involves some more damage to the host. Another disadvantage is that if the problem is local (or in this case the flow of infected fluid is low or almost null) then the method has to be applied directly to the affected area creating more undesirable damage. These methods, besides having several obvious drawbacks, can be many times more expensive than non-invasive ones for several reasons, and is because of this that alternatives to these approaches are highly desirable since it will have the capability to treat illnesses that affect populations with low resources.

In-vivo, direct pathogen (microorganisms) devitalization has been mentioned by Kronberg (U.S. Pat. No. 7,117,034) Apparatus and method for bioelectric stimulation, healing acceleration, pain relief, or pathogen devitalization. Describing a pulse width modulation type device which tailors signals for biomedical applications. The signals closely mirror natural body signals by operating at a signal level which is below the normal human threshold level of sensation and pain where most users do not experience any sensation during treatment apart from steady decrease in previously existing pain. The devices depicted have different applications among them the contemplation devitalization of organisms.

Finally, in the case of direct immune system activation and/or indirect pathogen load reduction, especially relevant art to the scope of this invention was done by Filipic et al. (Slovenia Pat. No. 9800214) Naprava in postopekza electrostimulaciyo celic—Proposes an in-vivo method for general immune activation and indirect viral inactivation by the induction of the release of antiviral substances from immunocompetent cells by the use of medium strength electric field pulses (0.5 to 500 V/cm) in a chamber that is part of an invasive dialysis-like system. And, in the non-invasive sense by Horiguchi et al. (U.S. Pat. Application No. 20070282391) Electric therapeutic appliance for improving immunity—Describes an electrotherapeutic device that is able to deliver a controlled and limited current of 1.5 to 4.5 uA trough some insulation to a hand(s) and/or foot (feet) by the use of a couple of plates that present high voltages between them (˜3000 to ˜9000 V) wherein chronic hepatitis viral infection diseases and human immunodeficiency virus chronic infection diseases are treated by increasing the number of immunocompetent cells.

### SUMMARY

Previous related art describes methods for direct pathogen load reduction, involved the exposure of bodily fluids to electromagnetic fields by the use of invasive methods and apparatuses, or in the case of the non-invasive approach they require the constant application of the electromagnetic field over a prolonged period of time to the same tissue where the pathogen is residing. The indirect methods for pathogen load reduction involve the activation and/or potentiation and/or normalization and/or regulation and/or stimulation and/or alertness and/or self tuning of the immune system or any of its parts or components by the use of an application system that required the use magnetic field magnitudes with a maximum of some mili-Teslas or a high voltage generator or more than one field in the same area (ICRF, NSR, etc.) or required of an invasive procedure.

There is then the need of an application capable of delivering electromagnetic fields inside of biological tissue with enough power to create an electric field with magnitudes in the order of 0.001 -500 V/cm to elicit these effects (activation and/or potentiation and/or normalization and/or regulation and/or stimulation and/or signaling and/or increase and/or regulation of the alertness and/or self tuning of the immune system) without the use of high voltage generators, any invasive procedure or the use of more than one field in the same area (neglecting the earth's magnetic field since the orientation with respect to that field would not be of significant relevance), that can be applied in conjunction with other therapies or procedures done in the living organism, that is cost effective, that is portable and that does not require or imply the generation or presence of high voltage.

Without conflicting with previous art, it is then the object of the present document to describe a method and a device that will be considered novel to any person skilled in the art. A Method and a Device that can deliver at lease one signal by means of electromagnetic fields non-invasively inside of biological tissue and/or biological fluid such as blood and/or lymph and/or synovial fluid and/or interstitial fluid and/or other fluids in-vivo and with magnitudes that do not cause or cause a tolerable amount of neural motor and sensory action potential generation to neural tissues by utilizing voltages below 50V RMS or 140V peak-to-peak and frequency components below the megahertz range by means of the placement of more than one electrode on the skin with or without some intermediate biological, synthetic, or natural agent or substance between the electrode and the skin and/or by induction by means of a time-varying magnetic field or both at the same time in different places of the body, having not necessarily the same signal, with the novel purpose of activation and/or potentiation and/or normalization and/or regulation and/or stimulation and/or signaling and/or regulate the alertness self tuning of the immune system or any of its parts or components directly or indirectly, locally and/or globally, with the purpose of pathogen load reduction and/or reduction of the disease symptoms and/or clinical improvement and/or tumor size reduction and/or malignancy reduction or amelioration and/or improvement of the effects of an autoimmune disorder or any other related condition that could be treated or remised by its effects, also presenting a optimized approach at the moment of the publication of this document.

### DESCRIPTION OF THE FIGURES

FIG. 1 is an exemplary schematic of the cross section of a part of a human or animal where the application of at least one of the time dependant signals by means of electric fields by the use of two electrodes that are in contact with an intermediate biological or synthetic or natural substance or agent that are in contact with the skin is taking place in accordance with at least some embodiments of the present invention.

FIG. 2 is an exemplary schematic of the cross section of a part of a human or animal where the application of at least one of the time dependant signals by means of magnetic fields by the use of a coil that is in contact with an intermediate biological or synthetic or natural substance or agent that is in contact with the skin is taking place in accordance with at least some embodiments of the present invention.

FIG. 3 is an exemplary schematic that shows the possible conjunct application of both exposure systems of the electrotherapeutic device that are applying not necessarily the same signal in accordance with at least some embodiments of the present invention.

FIG. 4 is an exemplary schematic describing the possible therapy sequences for at least one of the time dependant signals applied to the human or animal in accordance with at least some embodiments of the present invention.

FIG. 5 is an exemplary schematic of a possible application of at least one of the time dependant signals by means of electric fields as shown in FIG. 1.

FIG. 6 is an exemplary schematic of a possible configuration of the electrotherapeutic device in accordance with at least some embodiments of the present invention.

FIG. 7 is an exemplary schematic of a possible configuration of the system used to impersonate the configuration described in FIG. 6 for the generation of a signal comprised of pulses of a time dependant duty cycle and length being in its simplest form a biphasic square wave in accordance with at least some embodiments of the present invention.

FIG. 8 is an exemplary schematic of a possible application of at least one of the time dependant signals by means of n magnetic fields as shown in FIG. 2.

FIG. 9 is an exemplary schematic of a possible application of at least one of the time dependant signals by means of magnetic fields as shown in FIG. 2.

FIG. 10 is an exemplary schematic of a possible application of at least one of the time dependant signals by means of magnetic fields as shown in FIG. 2.

FIG. 11 is an exemplary schematic of a possible application of at least one of the time dependant signals by means of magnetic fields as shown in FIG. 2.

FIG. 12 is an exemplary schematic of a possible configuration of the system used to impersonate the configuration described in FIG. 6 for the generation of a signal comprised of pulses to be applied its simpler form or for the creation of a more complex signals by pulses of a time dependant duty cycle and length.

FIG. 13 is an exemplary schematic of one of the several possible physical realizations of the system described in FIG. 7.

FIG. 14 is an exemplary schematic of one of the several possible physical realizations of the system described in FIG. 12.

### DETAILED DESCRIPTION

The purpose of this description is to illustrate the general principles of the invention and should not be taken in a limiting sense.

It is then an object of the present document to describe a device and method that can deliver time-dependant signals non-invasively preferably but not limited to the interior of the blood and lymph vessels and other bodily fluids such as interstitial, synovial, and/or other fluid and/or tissue at the same time that the electric field parameters present within neural tissue do not cause or cause a tolerable amount of neural motor and sensory action potentials by means of electric fields by means of the placement of more than one electrode on the skin with or without some intermediate biological, synthetic, or natural agent or substance between the electrode and the skin, and/or by means of magnetic fields by means of at least one coil or alike with the novel purpose of activation and/or potentiation and/or normalization and/or regulation and/or stimulation and/or signaling and/or regulate the alertness and/or self tuning of the immune system and/or any of its parts or components directly or indirectly, locally or globally (by changing biochemical reaction rates, etc. and/or by changing the tissues' biophysical characteristics such as temperature, pH, etc. and/or by neural signaling and/or acupuncture-like stimuli, etc. and/or by activation or increase of the biochemical release by any of the exposed tissues or cells, etc. and/or by any other relevant mechanism of action or pathway not limited to the aforementioned) with the purpose of pathogen load reduction and/or reduction of the disease symptoms and/or clinical improvement and/or tumor size reduction and/or malignancy reduction or amelioration and/or improvement of the effects of an autoimmune disorder or any other related conditions that could be treated or remised by its effects.

The direct or indirect stimulation of immunocompetent cells by the signal applied which would generate a succession of events that would modulate entirely or in part the behavior of the immune system.

For example, when the electrotherapeutic device is applied animal or human which is infected with a viral infection disease in any of its forms i.e. chronic, acute, etc., the patient's immunity is activated to reduce the viral load and therefore the related symptoms.

When the electrotherapeutic device is applied to a patient infected with a human immunodeficiency virus (HIV), the patient's immunity is activated to reduce the viral load therefore the related symptoms.

When the electrotherapeutic device is applied to a patient infected with a dengue virus, the patient's immunity is activated to reduce the viral load therefore the related symptoms.

When the electrotherapeutic device is applied to a patient infected with a herpes virus, the patient's immunity is activated to reduce the viral load therefore the related symptoms.

When the electrotherapeutic device is applied to a patient infected with a hepatitis virus, the patient's immunity is activated to reduce the viral load therefore the related symptoms.

The set of signals generated will be time dependant and their parameters will be such that they will activate and/or potentiate and/or normalize and/or regulate and/or stimulate and/or signal and/or regulate the alertness and/or self-tuning of the immune system or any of its parts or components directly or indirectly, locally and/or globally with magnitudes comparable or higher than the natural noise generated by the target biological system and frequency components below the megahertz range.

In general, this signal can be expressed in this way:

$$\begin{matrix}s ( t ) = {\sum }_{i} {f}_{i} ( t - {τ}_{i} ) \\ For i = 0 , 1 , 2 … N \\ Where \text{:} & EQUATION 1 \\ \begin{matrix}{f}_{i} ( t ) = {g ( t )}_{i} [ u ( t ) - u ( t - ( {τ}_{i + 1} - {τ}_{i} ) ) ] \\ = \{ \begin{matrix}0 , & t < 0 \\ {g ( t )}_{i} , & 0 \leq t \leq ( {τ}_{i + 1} - {τ}_{i} ) \\ 0 , & t > ( {τ}_{i + 1} - {τ}_{i} )\end{matrix}\end{matrix} & EQUATION 2\end{matrix}$$

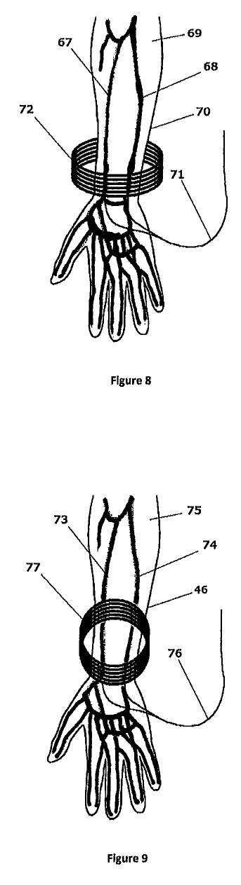

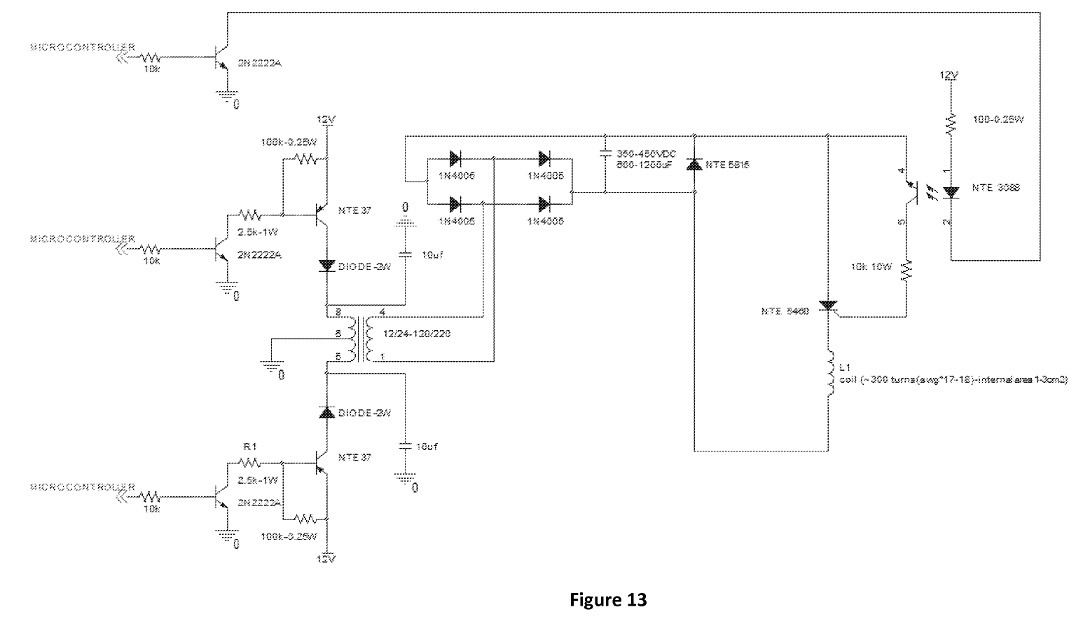

Being g(t)ᵢ a set of time dependant functions that are not necessarily equal, having as a result, a time-dependant frequency content.
The generation of one special case of s(t), if its parameters are chosen like so:

$${f}_{i} ( t ) = \{ \begin{matrix}0 , & t < 0 \\ {A}_{i} , & 0 \leq t \leq ( {τ}_{i + 1} - {τ}_{i} ) \\ 0 , & t > ( {τ}_{i + 1} - {τ}_{i} )\end{matrix}$$

Where Aᵢ is a set of constants For i=0,1,2 . . . N can be achieved by the generic and exemplary system shown in FIG. 7 where the 4 switches (61-65) are activated in pairs (61 and 65 or 62 and 63) being able to supply a biphasic chain of voltage pulses that are delivered to the electrodes or coil(s)—or other type of electric or magnetic transducer not limited to the ones available at the moment of the creation of this document—(64) being a able to ideally deliver twice the voltage that is supplied by the power conditioning and isolator(59) to the human or animal eliminating in that way any voltage multiplier or similar device or configuration making the power consumption used for the maintenance of the device minimal and maximizing in this way the power that is actually delivered to the transducers and under similar conditions to the human or animal. FIG. 7 generally depicts power conditioning and isolator (59) Voltage and current regulation and limiting (60) Switch in any of its forms (mechanic, semiconductor, etc.) (61)(62)(63)(65). Electrodes, applicators, conductors—for the first embodiment or Coil, applicator, conductors for the second embodiment.—or other type of electric or magnetic transducer not limited to the ones available at the moment of the creation of this document (64). Microcontroller, microprocessor, central processing unit, computer (66) User interface (89) power source (99). This configuration is preferably but not limited to be used when the power source is able to deliver instantaneously the power needed. Without limiting the scope of this invention, a simple example of the many different ways in which a physical realization of the generic and exemplary system shown in FIG. 7 circuit can be done is shown in FIG. 13.
The generation of another special case of s(t), is if its parameters are chosen like so:

$${f}_{i} ( t ) = \{ \begin{matrix}0 , & t < 0 \\ {E ( t )}_{i} , & 0 \leq t \leq ( {τ}_{i + 1} - {τ}_{i} ) \\ 0 , & t > ( {τ}_{i + 1} - {τ}_{i} )\end{matrix}$$

Where Eᵢ is a set of exponential decaying functions for i=0,1,2 . . . N which could be given by an expression such as:

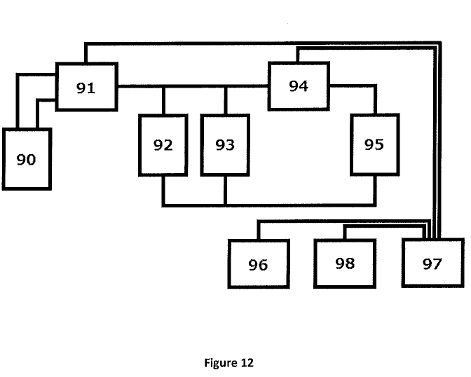

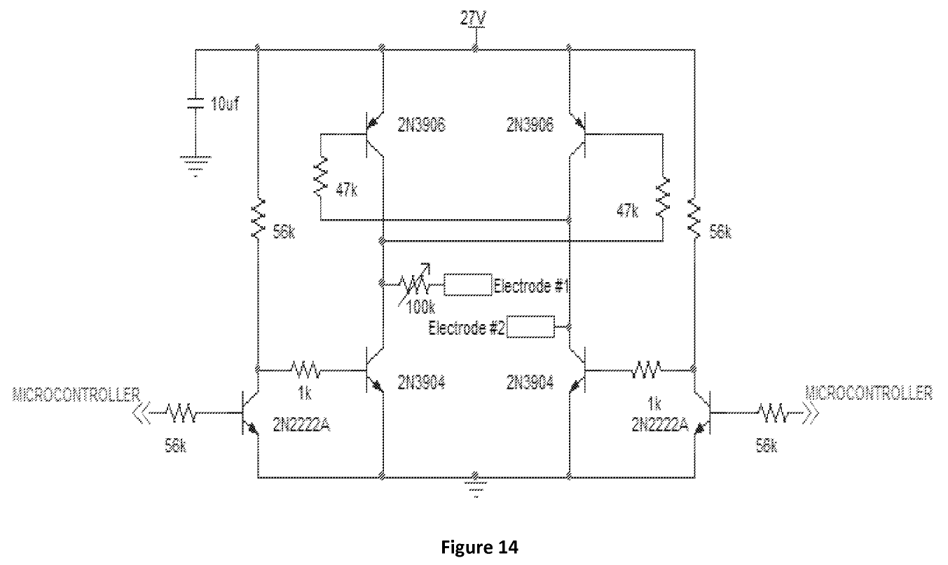

E(t)ᵢ=Bᵢe^{α}^{ᵢ}^{t}

Where Bᵢ is a set of functions and αᵢ is another set of functions for i=0,1,2 . . . N.
can be approximated by the generic and exemplary system shown in FIG. 12 where a unit for energy storage such as a capacitor (92) is charged from a power conditioning and isolator (91) so then that energy can be released to the electrodes or coil(s)—or other type of electric or magnetic transducer not limited to the ones available at the moment of the creation of this document—(95) by the activation a Switch in any of its forms (mechanic, semiconductor, etc.) (94) being able to deliver a large amount of energy to the electrodes or coil(s) in the case that this energy cannot be instantaneously delivered by the power source (90) or because of any other design limitation or specification. FIG. 12 generally depicts a Power source (90), Power conditioning and isolator (91), Energy storage device (capacitor etc.) overvoltage/voltage reversal protection (93), Switch in any of its forms (mechanic, semiconductor, etc.) (94), Electrodes, applicators, conductors—for the first embodiment or Coil, applicator, conductors for the second embodiment—or other type of electric or magnetic transducer not limited to the ones available at the moment of the creation of this document (95), User interface (96, Microcontroller, microprocessor, central processing unit, computer (97), and Storage memory in any of its forms (98). Two of this configurations (the one shown in FIG. 12) can also be used complementarily both as part of a configuration similar to the one shown in FIG. 7 in order to create a biphasic signal. Without limiting the scope of this invention, a simple example of the many different ways in which a physical realization of the generic and exemplary system shown in FIG. 12 is shown in FIG. 14.

Let the examples previously presented be interpreted differently as being a limit of the scope of this invention.

The electrotherapeutic device preferred embodiment will then be comprised of two different exposure systems (First exposure system—electric fields application, and Second exposure system—magnetic field application) in order to deliver the necessary electric field magnitude 0.001-500 V/cm inside of the biological tissue with a time-dependant signal that is below the megahertz range and that is not necessarily the same for both exposure systems.

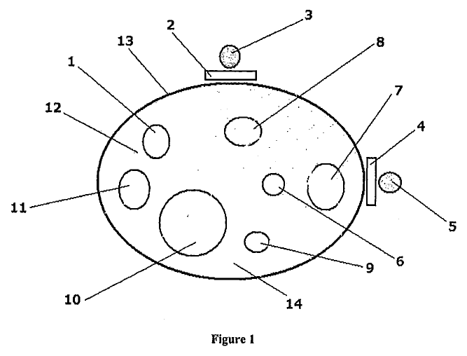

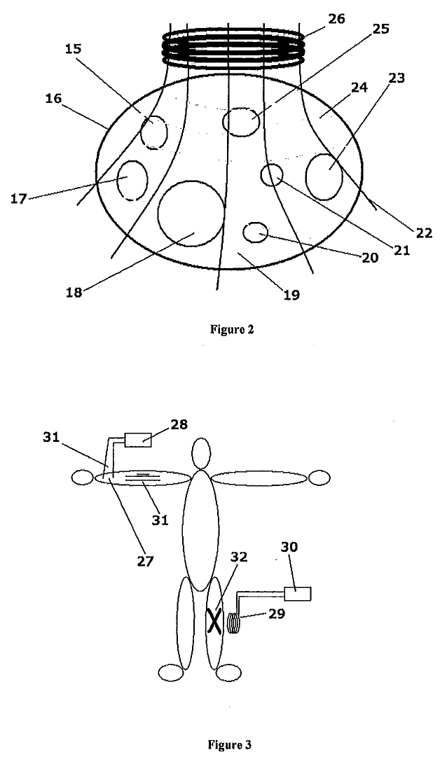

The scope of the invention includes the individual utilization of each exposure system for treatment, the utilization of both systems as part of the same treatment (FIG. 3) and the utilization of both systems during overlapping periods (FIG. 3) or at the same time with each other or with other therapy not described in this document each one with signals that are not necessarily the same. More specifically, FIG. 3 depicts a device capable of creating an electric field as shown in FIG. 1 (28), electrodes contacting the skin with an intermediate biological or synthetic or natural substance or agent (31), possible place of application of the device presenting 2 adjacent blood vessels (wrist)—ulnar and radial arteries—(27) treated liquids flowing to the rest of the body and untreated liquid flowing to the application site (31), Site of accumulation of infected fluid such as synovial fluid, lymph nodes, spleen, liver, etc (32). Coil for the delivery of magnetic fields (29) and Device capable of creating the necessary electric current to drive the coil to create an effect as shown in FIG. 2 (30)

### First Exposure System

The first Exposure System will deliver a time-dependant signal by means of electric fields with peak magnitudes within the previously defined range in the tissues of importance by utilizing more than one electrode (3,5) placed on the skin with or without some intermediate biological, synthetic, or natural agent or substance (2,4) between the skin and the electrode as shown in FIG. 1. The schematic of FIG. 1 represents generally and without loss of correspondence to a real human or animal part several tissues: Nerve (1), Vein (6), Artery (8), Lymph vessel (7), Bone (9), Fat (10), Synovial and/or other bodily fluids and/or tissue (11), Interstitial space (14), Equipotential field lines due to the applied electric field (12). This will be used in cases where the anatomy of the site of application in the biological entity allows for the necessary fields to get to the body fluids and other tissues of importance at the same time that the electric field parameters present within neural tissue do not cause or cause a tolerable amount of neural motor and sensory action potential.

The electrotherapeutic device when using the first exposure system will be comprised of more than one electrode (55) and a generation unit (57). Each electrode will be made of a conductive or partially conductive and relatively anticorrosive material and will be connected to the generation unit by a wire, flexible conductor or directly attached to the generation unit. The electrodes (3, 5,55) will make contact with the skin directly or indirectly by having an intermediate biological, synthetic or natural agent or substance or a composition of the previously mentioned (2,4). Some examples of this would be conductive gel, cotton soaked with a conductive liquid such as water with an electrolyte, etc. The electrodes will be held against the skin by its own means (for example suction, etc.) or by the use of any holding aid such as an elastic band, adhesive tape, etc. The electrodes can also be grabbed by the hands or the human or animal can stand over, lie, sit, hang, etc. on/over/from the electrodes. The generation unit (57) will generate the signal fed to the electrodes (55). The signal s(t) will be tailored by the combination of a set of functions fᵢ(t) which will have frequency components below the megahertz range and which will be generated and/or pre processed and/or post processed and/or controlled by a microcontroller, microprocessor, computer or any other digital device (50) which will be aided by a storage memory in any of its forms but not limited to the available at the moment of the filing of this document (51) and will be also power amplified (52) and filtered (53) and the system can also be aided of feedback control loops such as a current monitoring circuit, etc.

The voltages generated by the device at the electrodes will be considered as Extra Low Voltage (below 50V RMS or 140V peak-to-peak) according to the International Electrotechnical Commission. A current limiting circuit can also be implemented to limit the output current to the electrodes and in the case of wrist application (FIG. 5) the maximum threshold will be of less than 100 mA.

This exposure system would be very likely to be preferred when the target tissue characteristics in general allows for this method to be used in order to reach the aforementioned field magnitudes within that tissue.

FIG. 5 generally depicts an electrode (48), intermediate biological or synthetic or natural substance or agent (42), skin (46) ulnar artery (44), radial artery (43) Human arm(45) insulated electrical conductor(47). FIG. 5 shows a possible way in which electrodes can be applied on the skin over the ulnar and radial arteries in order to, for example, target arterial blood and tissue in that area. If blood is the target then the electric field generated in the chosen portion of the human or animal body will target only a part of the total amount of blood at a given moment, but since the blood will circulate in a relatively short period of time then eventually most of the blood will circulate through the area where the field is applied. The approach shown in FIG. 5 is also useful if the target tissue is the nerves below the wrist which could also be stimulated for eliciting an immune response with the same objective.

Let this example not to be a limit of the scope of the invention as this approach can be adapted for use in the arm, leg or foot or any combination of these, or any other part of the human or animal body Let this example not to be limited to the aforementioned materials or methods available at the moment of the creation of this document for the physical realization of the exposure system.

A way to calibrate the electric field magnitude when the electrodes are applied on the human wrist as shown in FIG. 5, for example, is to use the motor neural tissue as a sensor. This means that since there is a maximum in the parameters of a time-varying electric field such as magnitude (which depends on the specific waveform) that will trigger a motor action potential, (i.e. that will create movement on the hand) or pain, that magnitude can be varied in order to be right below the generation of this movement. For this, the electrotherapeutic device presents a dial which allows the user to vary the voltage magnitude of the waveform after its generation in the device or this is done automatically with the aid of a control loop. This is important since the electrode distance and position will vary between applications and between users due to obvious anatomical differences. For example, let be the case of two different users with exactly equal characteristics except size and a fairly steady signal that is being applied to the electrodes. User #1 has a wider wrist than user #2 i.e. user #2's wrist will be a miniaturized version of user #1's wrist. The maximum electric field magnitude between both electrodes in user #1 will be smaller in principle than user #2's and therefore the maximum electric field magnitude delivered to the neural tissue will also be smaller. This means that if user #1 shows no motor action potentials (movement in his or her hand) but is in the threshold, and if all the assumptions done previously prevail, then user #2 will show movement which is undesired and will have to use the dial to change the magnitude of the voltage applied in order to decrease the field magnitude to a point where no movement is longer observed. The same situation will happen when the electrodes position or contact with the skin or users skin or tissue conductivity varies, and the dial in conjunction with the feedback provided by the hand movement will help to set the device to the maximum possible output magnitude.

Being the previous explanation a general example of one of the ways in which the output amplitude of the device can be set, let not be this a limiting factor for possible analogous designs under the same scope of this invention.

Another example of first exposure system is the use of electrodes that can be held by the subject in both hands. This electrodes could have a cylindrical or other shape—being this shape not limiting in any sense to the scope of the invention—and could have or not some intermediate biological, synthetic, or natural agent or substance between the electrode and the hand such as in the form of a hood or covering.

Another example of first exposure system is the use of electrodes in the shape of adjustable size or elastic band. This electrode can be worn in any part of the limbs and torso and any combination between these parts. For example, in the case of using 2 electrodes, some combinations could be: form wrist to wrist, from heel to heel, from heel to wrist, etc.

Another example of first exposure system is the use of electrodes that will allow the human or animal to stand, walk, sit or lie or hang on/over/from them in a way that contact can be done in more than one place and could have or not some intermediate biological, synthetic, or natural agent or substance between the electrodes and the skin.

Let the scope of the invention not be limited by only the examples presented and its combinations.

### Second Exposure System

The second Exposure System will deliver a time-dependant signal by means of magnetic fields with peak magnitudes within the previously defined range in the tissues of importance by utilizing a at least one coil as shown in FIG. 2. This will be used in cases where the anatomy of the site of application in the biological entity allows for the necessary fields to get to the body fluids and other tissues at the same time that the electric field parameters present within neural tissue do not cause or cause a tolerable amount and sensory action potential. More specifically, FIG. 2 shows a schematic that generally represent and without loss of correspondence to a real human or animal part, several tissues: Nerve (15), Vein (17), Artery (18), Lymph vessel (20), Bone (21), Fat (25), synovial and/or other bodily fluids and/or tissue (23), Interstitial space (19), Equipotential field lines due to the applied time-varying magnetic field (24) Magnetic flux lines (22).

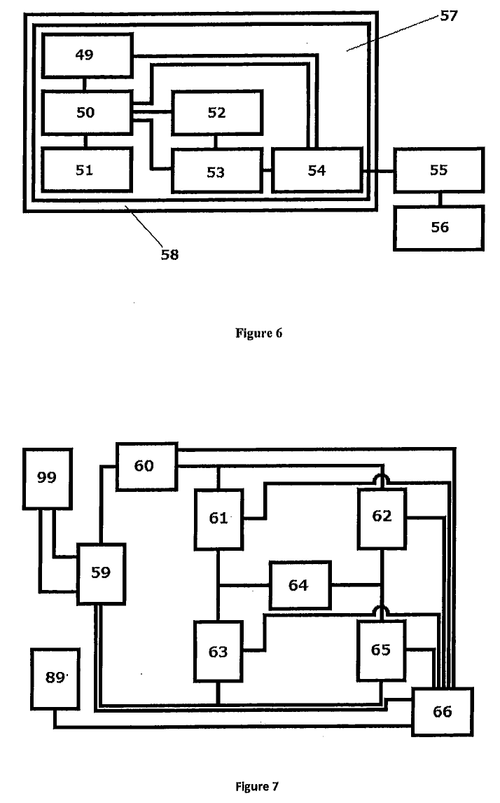

FIG. 6 depicts an exemplary microcontroller, microprocessor, central processing unit, computer (50). Storage memory in any of its forms (51). User interface (49). Amplification (52). Filtering (53). Current/voltage limiting (54) Electrodes, applicators, conductors—for the first embodiment or Coil, applicator, conductors for the second embodiment or other type of electric or magnetic transducer not limited to the ones available at the moment of the creation of this document (55). User (56) Generation Unit (57). Battery power and safety circuitry (58). The electrotherapeutic device when using the second exposure system may comprise a coil(s) (55) and a generation unit (57) (FIG. 6) where the coil(s) are made of a conductive material and connected to the generation unit by a wire, flexible conductor or directly attached to the generation unit(57). The coil(s) will be in contact with the skin directly or indirectly by having an intermediate biological, synthetic or natural agent or substance or a composition of the previously mentioned which will be non-conductive and/or the coil(s) will be applied at a defined distance of the skin. The coil(s) will be held in position by the user, the therapist, an elastic or adjustable belt or by a mechanical device for this purpose. The generation unit (57) will generate the signal fed to the coil(s). The signal s(t) will be tailored by the combination of a set of functions fᵢ(t) which will have frequency components below the megahertz range and which will be generated and/or pre processed and/or post processed and/or controlled by a microcontroller, microprocessor, computer or any other digital device (50) which will be aided by a storage memory in any of its forms but not limited to the available at the moment of the filing of this document (51) and will be also power amplified (52) and filtered (53) and the system can also be aided of feedback control loops such as a current monitoring circuit, etc.

FIG. 12 depicts a possible configuration of the generalized system depicted in FIG. 6. More specifically, FIG. 12 depicts a Power source (90), Power conditioning and isolator (91), Energy storage device (capacitor etc.) overvoltage/voltage reversal protection (93), Switch in any of its forms (mechanic, semiconductor, etc.) (94), Electrodes, applicators, conductors—for the first embodiment or Coil, applicator, conductors for the second embodiment or other type of electric or magnetic transducer not limited to the ones available at the moment of the creation of this document (95), User interface (96), Microcontroller, microprocessor, central processing unit, computer (97), and Storage memory in any of its forms (98).

The magnetic field peak magnitude generated by the device at the center of the coil(s) will be greater than 0.01 Tesla. This, together with an adequate rate of change of the magnetic field, will generate an electric field of the aforementioned characteristics.

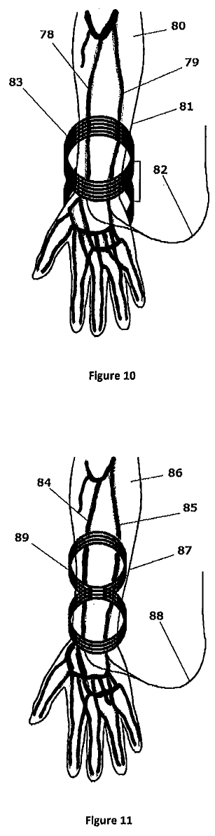

Although this approach can be used in the same way than the first exposure system (FIG. 8, FIG. 9, FIG. 10, FIG. 11), this approach would be very likely to be preferred when the target tissue is not easily reachable with the first exposure system under the restrictions previously mentioned, complementing the first exposure system. An electromagnetic field will penetrate and if not static, generate a time-varying electric field in the tissue needed to achieve similar effects than for the first described device. FIG. 8 generally depicts a radial artery (67), ulnar artery (68), Human arm (69), skin (70), insulated electrical conductor (71), and Coil (72). FIG. 9 generally depicts a radial artery (73), ulnar artery (74), Human arm (75), skin (76) insulated electrical conductor (77), and Coil (78). FIG. 10 generally depicts a radial artery (79), ulnar artery (80), Human arm (81), skin (82) insulated electrical conductor (83), and Coil (84). FIG. 11 generally depicts a radial artery (85), ulnar artery (86), Human arm (87), skin (88), insulated electrical conductor (89), and Coil (90).

FIG. 3 shows an example of how both exposure systems can be used at the same time or at overlapping treatment periods. An electrotherapeutic device equipped with the first exposure system is used to target arterial blood and other tissue and fluid at the same time that another electrotherapeutic device equipped with the second exposure system is used to target synovial fluid and/or other tissue and/or fluid while both devices are not applying not necessarily the same signal.

Let this example not to be a limit of the scope of the invention as this approach can be adapted for use in any other part of the body.

An example of second exposure system is the use of one coil surrounding the area where the target tissue resides (FIG. 8). Another example of second exposure system is the use of a coil outside of the part of the body where the tissue of interest resides (FIG. 9). Another example of second exposure system is the use of two complementary coils outside of the part of the body where the tissue of interest resides (FIG. 10). Two complementary coils will create a more uniform field than the field created by the previous examples. Another example of second exposure system is the use of a figure-8 coil outside of the part of the body where the tissue of interest resides (FIG. 11). A coil as depicted in FIG. 8 may be used to create a field that is mainly perpendicular to the field that can be created by the other examples.

It should be appreciated that the scope of the invention is not limited by only the examples here presented and its combinations.

### Method of Application

The method for the application of a treatment with at least one of the signals generated by any of the electrotherapeutic devices' and applied by its exposure systems will be comprised of 3 different cycles. The time from an application period and a rest period will add to make a micro-cycle. For example, if the application period is 1 hour out of every 12 (11 hours of rest period) then the micro-cycle will be of 12 hours. Micro-cycles of the same length or Micro-cycles of different lengths can group together (with or without rests periods of same or variable length between them) to form macro-cycles and macro-cycles can be combined in a similar fashion to form mega-cycles. (FIG. 3)

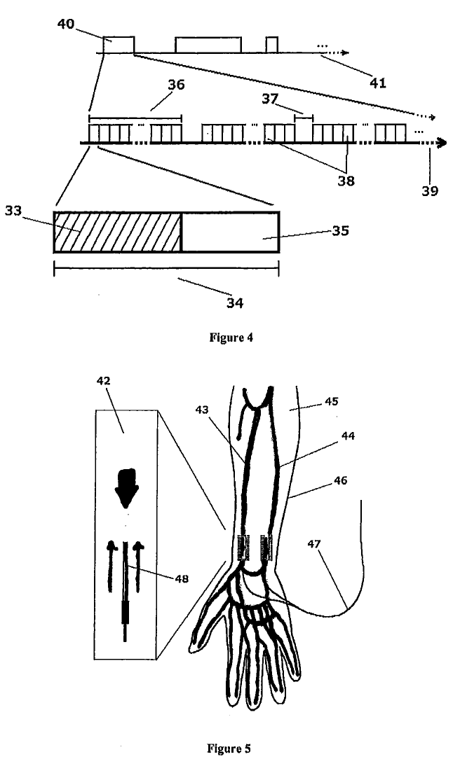

FIG. 4 depicts a variable application period (33) and a resting period (35) will form a basic block: a micro-cycle (36) one or more than one micro-cycles combined with variable resting period(s) (37) will be another basic building block: a macro-cycle (40) One or more than one macro-cycles combined with variable length resting period(s) will create a complete possible treatment period (Mega-cycle) (41).

It is also the object of the invention to describe the utilization of both exposure systems as part of the same treatment. It is also the object of the invention the utilization of both exposure systems during overlapping periods or at the same time with each other or with other therapy not described in this document.

### EXAMPLE OF APPLICATION

### EXAMPLE 1

Possible application for Lymphatic Filariasis, known as Elephantiasis, It is clear that this anti-parasite treatment can result in improvement of patients' elephantiasis and hydrocoele (especially in the early stages of disease), but the most significant treatment advance to alleviate the suffering of those with elephantiasis has come from recognizing that much of the progression in pathology results from bacterial and fungal “superinfection” of tissues with compromised lymphatic function caused by earlier filarial infection. Thus the application of the invention would be of much help as an adjunctive measure to minimize infection and promote lymph flow, which will result both in a reduction in frequency of acute episodes of inflammation (“filarial fevers”) and in a degree of improvement of the elephantiasis itself. The treatment could be comprised of the two exposure systems at the same time not necessarily with the same signals. The first exposure system electrodes would be placed, for example, on the wrist with the purpose of activation and/or potentiation and/or normalization and/or regulation and/or stimulation and/or signaling and/or regulate the alertness and/or self-tuning of the immune system or any of its parts or components directly or indirectly, locally and/or globally with the purpose of pathogen load reduction and/or reduction of the disease symptoms and/or clinical improvement and/or amelioration of any other related condition that could be treated or remised by its effects. The second exposure system could be utilized over the endemically affected areas that present swallowing and deformity due to the low flow of fluids such as lymph in those areas and the possible difficulty to reach them with the other method.

### EXAMPLE 2

Knee inflammation due to synovial and proximal liquid and/or tissue infection patient: The first exposure system (electrodes) are placed on the skin over the radial and ulnar arteries at the same time as the second exposure system(coil) is placed over or near the knee area. The preferred micro-cycle is of 23 hours rest by 1 hour of application. The micro-cycle is repeated until the symptoms retreat. Symptoms retreated after 3 days of application.

### EXAMPLE 3

HIV/AIDS patient: The first exposure system with a micro-cycle of 11 hours of rest by 1 hour of application is chosen. The micro cycle is applied during 20 days and then a rest of another 20 days. The measured viral load corresponds to less than 60% of the original sample before the treatment.

The present invention, in various embodiments, includes components, methods, processes, systems and/or apparatus substantially as depicted and described herein, including various embodiments, subcombinations, and subsets thereof. Those of skill in the art will understand how to make and use the present invention after understanding the present disclosure. The present invention, in various embodiments, includes providing devices and processes in the absence of items not depicted and/or described herein or in various embodiments hereof, including in the absence of such items as may have been used in previous devices or processes, e.g., for improving performance, achieving ease and/or reducing cost of implementation.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments for the purpose of streamlining the disclosure. This method of disclosure is not to be interpreted as reflecting an intention that the claimed invention requires more features than are expressly recited in each, claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

Moreover, though the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method, comprising:
generating at least one time-dependant signal;
applying, non-invasively, the generated signal(s) by means of electric and/or magnetic fields to a human or animal in a way that the existing applied or induced electric field(s) in the interior of the human or animal remain within magnitudes in the order of about 0.001 V/cm to about 500 V/cm.

2. The method of claim 1, wherein the voltages applied remain below 50V RMS or 140V peak-to-peak and wherein the electric field is applied and/or induced to at least one of an interior of the blood and lymph vessels and other bodily fluids and tissue of the human or animal at the same time that the time-varying electric field present within neural tissue is below what is needed for the generation of motor and/or uncomfortable or painful sensory action potentials.

3. The method of claim 1, wherein the signal applied comprises frequencies below the megahertz range.

4. The method of claim 1, wherein the signal is applied over at least one of an ulnar and radial artery of the human or animal.

5. The method of claim 1, wherein the signal is applied via a least one electrode placed on or near the skin of the human or animal with or without an intermediary material between at least one electrode and the skin.

6. The method of claim 5, to a human and wherein the at least one electrode is held by the human in at least one hand with or without an intermediary material between at least one electrode and the skin.

7. The method of claim 5, wherein the at least one electrode comprises an adjustable band.

8. The method of claim 1, wherein the human or animal is allowed to at least one of stand, walk, sit, or lay down or hang from the electrodes while at least one of the signals is applied thereto.

9. The method of claim 1, wherein the signal is applied to a multicellular organism.

10. The method of claim 1, wherein at least one of the signals is applied via at least one coil.

11. The method of claim 10, wherein the magnetic field magnitude applied by the at least one coil is above 100 Gauss.

12. An apparatus, comprising:
a signal generator operable to generate a at least one time-dependant signal; and
at least one electrode that is operable to apply, non-invasively, the generated signal to a human or animal by means of electric fields by means of the placement of more than one electrode on the skin of the human or animal with or without an intermediary material between at least one electrode and the skin in order to create a time-varying electric field in the interior of the human or animal with magnitudes in the order of about 0.001 V/cm to about 500 V/cm.

13. The apparatus of claim 12, wherein the voltage delivered to the electrodes remain below 50V RMS or 140V peak-to-peak and wherein the signal is applied to at least one of an interior of the blood and lymph vessels and other bodily fluids and tissue of the human or animal at the same time that the electric field magnitude present within neural tissue is below what is needed for the generation of motor and/or uncomfortable or painful sensory action potentials.

14. The apparatus of claim 12, wherein the signal applied comprises frequencies below the megahertz range.

15. The apparatus of claim 12, wherein the signal is applied on the skin over at least one of an ulnar and radial artery of the human or animal.

16. The apparatus of claim 12, wherein the signal is applied to a human and wherein the at least one electrode is held by the human in both hands.

17. The apparatus of claim 12, wherein the at least one electrode comprises an adjustable band.

18. The apparatus of claim 12, wherein the signal is applied to multicellular organism.

19. An apparatus, comprising:
a signal generator operable to generate a at least one time-dependant signal; and
at least one coil that is operable to apply, non-invasively, the generated signals to a human or animal by means of magnetic fields by means of the placement of at least one coil within a predetermined distance of the human or animal.

20. The method of claim 19, wherein the magnetic field magnitude generated by the at least one coil is above 100 5 Gauss, wherein the signals utilized will be composed of frequencies below the megahertz range, wherein a time-varying magnetic fields will induce time-varying electric fields in the interior of the human or animal with magnitudes in the order of about 0.001 V/cm to about 500 V/cm, and wherein the signal is applied to multicellular organism.
